(19) United States
(12) Patent Application Publication
(10) Pub. No.: US 2023/0263648 A1
(43) Pub. Date: Aug. 24, 2023

(54) DRUG ELUTION FOR IMPLANTABLE MEDICAL DEVICE

(71) Applicant: Boston Scientific Scimed, Inc., Maple Grove, MN (US)

(72) Inventors: Andrew Roiko, Rogers, MN (US); Brady Scott Logan, Monticello, MN (US)

(73) Assignee: Boston Scientific Scimed, Inc, Maple Grove, MN (US)

(21) Appl. No.: 18/112,114

(22) Filed: Feb. 21, 2023

Related U.S. Application Data
(60) Provisional application No. 63/312,221, filed on Feb. 21, 2022.

(51) Int. Cl. A61F 2/90; A61F 2/962

(52) U.S. Cl. CPC A61F 2/90; A61F 2/962; A61F 2250/0067

(57) ABSTRACT

A delivery device is adapted to deliver an implantable medical device to a deployment site and includes an elongate shaft extending from a proximal region to a distal region. The elongate shaft defines a first portion adapted to accommodate the implantable medical device, and a second portion adapted to accommodate a drug eluting component to be delivered along with the implantable medical device. In some cases, an implantable medical device includes an expandable body and one or more drug eluting components.

### CROSS-REFERENCE TO RELATED APPLICATIONS

This application claims the benefit of priority under 35 U.S.C. § 119 of U.S. Provisional Application No. 63/312,221, filed Feb. 21, 2022, the entire disclosure of which is hereby incorporated by reference.

### TECHNICAL FIELD

The present disclosure pertains to medical devices, and methods for manufacturing and using medical devices.

### BACKGROUND

The present disclosure pertains to medical devices, and methods for manufacturing medical devices. More particularly, the present disclosure pertains to intracorporeal medical devices, and methods for manufacturing and using such devices. Of the known medical devices and methods, each has certain advantages and disadvantages.

### SUMMARY

This disclosure provides design, material, manufacturing method, and use alternatives for medical devices. As an example, a delivery device may be adapted to deliver an implantable medical device to a deployment site. The delivery device includes an elongate shaft extending from a proximal region to a distal region. The elongate shaft includes a first portion that is adapted to accommodate the implantable medical device, and a second portion that is adapted to accommodate a drug eluting component to be delivered along with the implantable medical device. The delivery device is adapted to release the implantable medical device at the deployment site.

Alternatively or additionally, the second portion may include a passageway in fluid communication with a source of an elutable drug.

Alternatively or additionally, the delivery device may be adapted to bathe the implantable medical device with the elutable drug prior to releasing the implantable medical device.

Alternatively or additionally, the delivery device may be adapted to bathe the deployment site with the elutable drug either before, during or after releasing the implantable medical device.

Alternatively or additionally, the second portion may keep the drug eluting component away from the implantable medical device until deployment.

Alternatively or additionally, a sheath may separate the first portion from the second portion until the sheath is retracted.

Alternatively or additionally, the drug eluting component may include one or more elongate members carrying an elutable drug.

Alternatively or additionally, the one or more elongate members may include threads or wires that are impregnated or coated with the elutable drug.

As another example, an implantable medical device includes a body that is expandable from a compressed delivery configuration to an expanded deployment configuration. One or more drug eluting components are formed separately from the body and are disposed proximate the body upon deployment of the implantable medical device.

Alternatively or additionally, the one or more drug eluting components may be woven into the body.

Alternatively or additionally, the one or more drug eluting components may be dispersed over an exterior of the body.

Alternatively or additionally, the one or more drug eluting components may be disposed proximate the body after the body has been compressed into its compressed delivery configuration.

Alternatively or additionally, the one or more drug eluting components may be delivered proximate the body after the body has been delivered and before the body has expanded into the expanded deployment configuration.

Alternatively or additionally, the implantable medical device includes a stent.

Alternatively or additionally, the implantable medical device includes a cardiac implant.

As another example, a bare metal stent includes an expandable elongate body that is expandable from a compressed delivery configuration to an expanded deployment configuration. One or more drug eluting components are formed separately from the expandable elongate body and are disposed proximate the expandable elongate body upon deployment of the bare metal stent.

Alternatively or additionally, the one or more drug eluting components may be woven into the expandable elongate body.

Alternatively or additionally, the one or more drug eluting components may be dispersed over an exterior of the expandable elongate body.

Alternatively or additionally, the one or more drug eluting components may be disposed proximate the expandable elongate body after the expandable elongate body has been compressed into its compressed delivery configuration.

Alternatively or additionally, the one or more drug eluting components may be delivered proximate the expandable elongate body after the expandable elongate body has been delivered and before the expandable elongate body has expanded into the expanded deployment configuration.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic cross-sectional view of a portion of an illustrative delivery device;

FIG. 2 is a side view of an illustrative drug eluting component;

FIG. 3A is a schematic cross-sectional view taken along line 3-3 of FIG. 2;

FIG. 3B is a schematic cross-sectional view taken along line 3-3 of FIG. 2;

FIG. 4 is a schematic cross-sectional view of a portion of an illustrative delivery device;

FIG. 5 is a side view of an illustrative implantable medical device including a plurality of drug eluting components;

FIG. 6 is a side view of an illustrative implantable medical device including a plurality of drug eluting components;

FIG. 7 is a side view of an illustrative implantable medical device including a drug eluting component extending within the implantable medical device;

FIG. 8 is a side view of an illustrative implantable medical device including a pair of drug eluting components woven into the implantable medical device; and

FIG. 9 is a side view of an illustrative implantable medical device including a plurality of drug eluting components.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

There are a number of different types of implantable medical devices that may benefit from adding a possibility of elutable drugs to the implantable medical devices. In some instances, an implantable medical device is moveable between a compressed configuration and an expanded configuration, for example. In some cases, it may be difficult to simply coat an implantable medical device with one or more elutable drugs due to the materials that the implantable medical device is made of, for example, or because of relative movement between parts of the implantable medical device when the implantable medical device is delivered and deployed. As an example, many stents such as knitted stents and braided stents are formed from one or more filars that are knitted or braided together in order to form the body of the stent. It will be appreciated that the filars move relative to each other when the stent moves from a compressed configuration to an expanded configuration. If the elutable drug is simply coated onto the filars, this relative movement may cause at least some of the elutable drug to wear off of the stent before the stent can even be delivered and deployed.

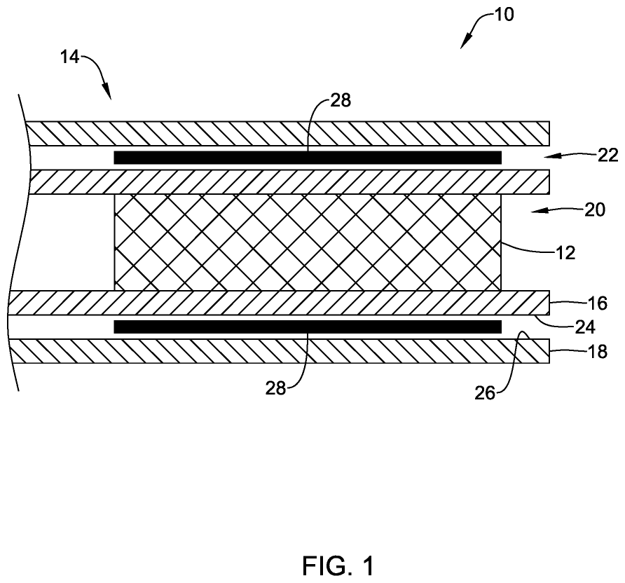

FIG. 1 is a schematic view of a distal region of an illustrative delivery device 10 that may be used to deliver an implantable medical device 12. While the implantable medical device 12 is shown as a stent, it will be appreciated that any of a variety of different implantable medical devices 12 may be delivered via the delivery device 10. The delivery device 10 includes an elongate shaft 14. In some instances, the elongate shaft 14, or at least the distal region thereof, may include an inner tubular member 16 and an outer tubular member 18. The inner tubular member 16 defines a lumen 20 that may be considered as being adapted to accommodate the implantable medical device 12 within the lumen 20. In some cases, the implantable medical device 12 may be deployed by withdrawing the inner tubular member 16 proximally relative to the implantable medical device 12. The implantable medical device 12 may be held against a stop formed by another feature (not shown) of the delivery device 10. In some cases, the implantable medical device 12 may be deployed by using a pusher or other elongate member (not shown) to push the implantable medical device 12 distally relative to the inner tubular member 16, thereby deploying the implantable medical device 12 out of the distal end of the delivery device 10.

In some instances, as shown, the outer tubular member 18 may form an annular lumen 22 between an outer surface 24 of the inner tubular member 16 and an inner surface 26 of the outer tubular member 18. In some instances, the annular lumen 22 may be adapted to accommodate one or more drug eluting members 28 within the annular lumen 22. While a pair of drug eluting members 28 are shown in this two-dimensional representation of the delivery device 10, it will be appreciated that since the annular lumen 22 may extend 360 degrees circumferentially about the delivery device 10, the annular lumen 22 may accommodate any desired number of drug eluting members 28. For example, there may be a total of four drug eluting members 28, each spaced circumferentially about 90 degrees apart. There may be a total of eight drug eluting members 28, each spaced circumferentially about 45 degrees apart. These are just examples.

In some cases, the drug eluting members 28 may be released from the annular lumen 22 by withdrawing the outer tubular member 18 proximally relative to the drug eluting members 28. In some cases, the drug eluting members 28 may be pushed distally out of the annular lumen 22 via a pusher (not shown). In some cases, the drug eluting members 28 may be released from the delivery device 10 at the same time that the implantable medical device 12 is released. The drug eluting members 28 may be released after the implantable medical device 12 is released. In some instances, the drug eluting members 28 may be held in position by being pinned between the implantable medical device 12 and a side wall of a lumen in which the implantable medical device 12 is deployed.

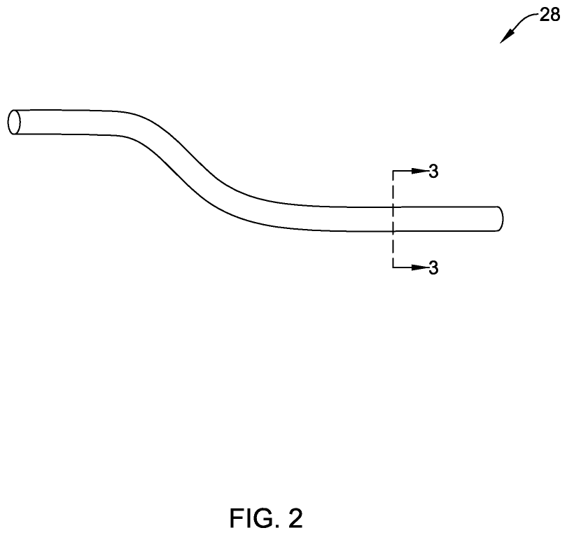

The drug eluting members 28 may take a variety of forms. FIG. 2 is a side view of one of the drug eluting members 28. The drug eluting member 28 may be formed of any of a variety of different materials, such as a polymer or a metal. The drug eluting member 28 may be a wire or a thread. The drug eluting member 28 may be a metal wire or a thread, for example, and may have a diameter that is in the range of about 0.01 inches to about 0.2 inches, or a diameter that is in the range of about 0.05 inches to about 0.015 inches, or perhaps a diameter that is in the range of about 0.075 inches to about 0.0125 inches. These are just examples, and other diameters are also contemplated.

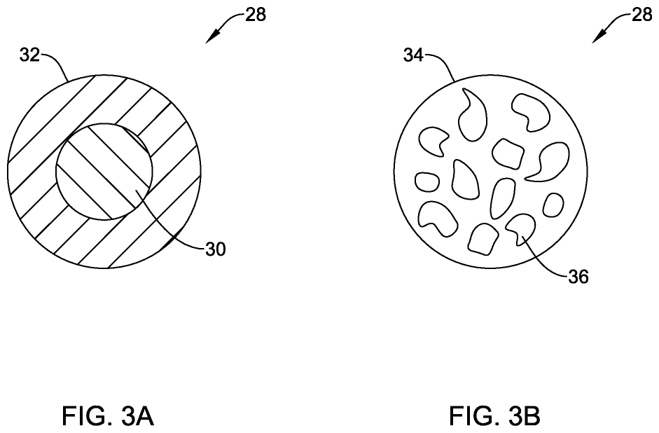

In some cases, the drug eluting member 28 may include a core 30 and a drug layer 32 that is disposed about the core 30, as shown in FIG. 3A. The core 30 may be a polymer or a metal, for example. The drug layer 32 may be dip coated or spray coated onto the core 30. The drug layer 32 may include one or more therapeutic drugs that are dispersed within a carrier such as a polymer layer. The drug layer 32 may include one or more therapeutic drugs that are directly coated onto the core 30 without a carrier. The drug eluting member 28 may have a length that is selected to be at least as long as a major dimension of the implantable medical device 12, for example. Any of a variety of therapeutic drugs may be used. Illustrative examples include paclitaxel and derivatives thereof, everolimus and derivatives thereof, rapamycin (sirolimus) and derivatives thereof, heparin and others.

In some cases, as shown for example in FIG. 3B, which is another schematic cross-section taken of the drug eluting member 28, the drug eluting member 28 may be formed of a porous member 34 having a plurality of apertures or voids 36 that are filled with one or more therapeutic drugs. In some cases, the porous member 34 may be considered as providing a framework or structure, defining the plurality of apertures or voids 36, that may hold a plurality of microspheres that are formed of or otherwise include one or more therapeutic drugs.

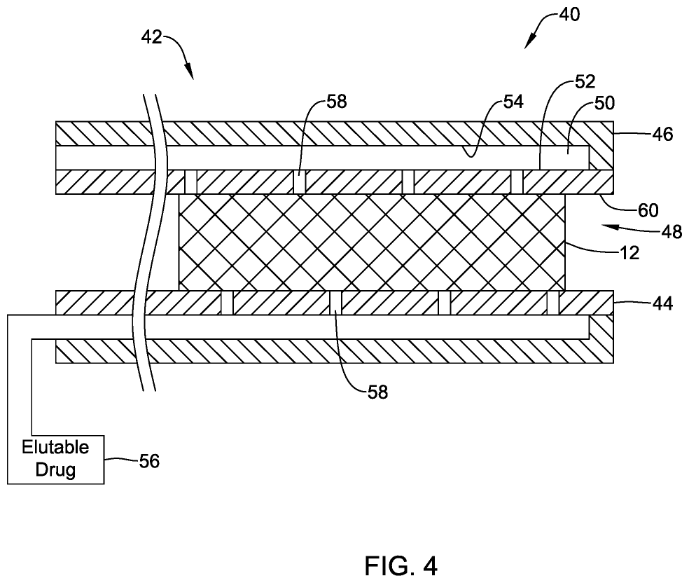

FIG. 4 is a schematic view of an illustrative delivery device 40 that may be used to deliver the implantable medical device 12. While the implantable medical device 12 is shown as a stent, it will be appreciated that any of a variety of different implantable medical devices 12 may be delivered via the delivery device 40. In some cases, the implantable medical device 12 is a bare metal stent. The delivery device 40 includes an elongate shaft 42. In some instances, the elongate shaft 42, or at least a distal region thereof, may include an inner tubular member 44 and an outer tubular member 46. The inner tubular member 44 defines a lumen 48 that may be considered as being adapted to accommodate the implantable medical device 12 within the lumen 48. In some cases, the implantable medical device 12 may be deployed by withdrawing the inner tubular member 44 proximally relative to the implantable medical device 12. The implantable medical device 12 may be held against a stop formed by another feature (not shown) of the delivery device 40. In some cases, the implantable medical device 12 may be deployed by using a pusher or other elongate member (not shown) to push the implantable medical device 12 distally relative to the inner tubular member 44, thereby deploying the implantable medical device 12 out of the distal end of the delivery device 40.

In some instances, as shown, the outer tubular member 46 may form an annular lumen 50 between an outer surface 52 of the inner tubular member 44 and an inner surface 54 of the outer tubular member 46. In some instances, the annular lumen 50 may be adapted to be in fluid communication with a source 56 of an elutable drug. The source 50 of the elutable drug may include one or several different therapeutic drugs, for example. The one or several different therapeutic drugs may be suspended or otherwise combined with a suitable liquid carrier such as saline.

The inner tubular member 44 includes a number of voids 58 that each extend from the outer surface 52 of the inner tubular member 44 to an inner surface 60 of the inner tubular member 44. The voids 58 provide for fluid communication from the source 56 of elutable drug, through the annular lumen 50 and into the lumen 48. Consequently, the delivery device 40 can be used to bathe the implantable medical device 12 with one or more therapeutic drugs prior to deploying the implantable medical device 12. In some cases, the delivery device 40 can be used to bathe a deployment site within one or more therapeutic drugs either during or even after deployment of the implantable medical device 12.

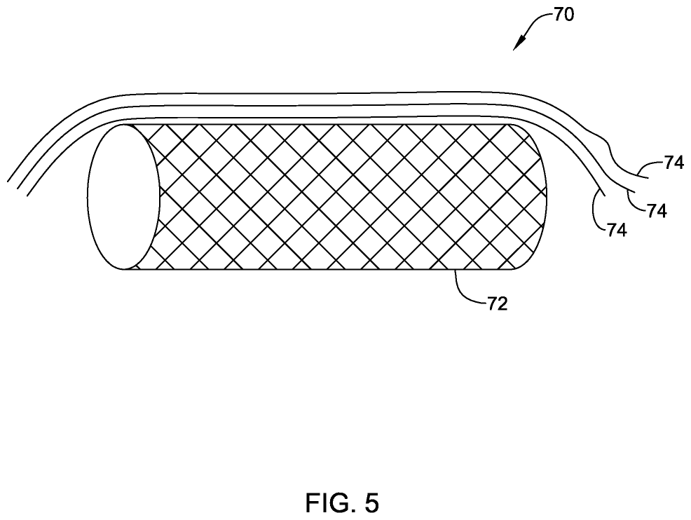

FIG. 5 is a side view of an illustrative implantable medical device 70. The implantable medical device 70 includes an elongate expandable body 72 that is moveable between a compressed configuration for delivery (not shown) and an expanded configuration, as illustrated. The implantable medical device 70 includes several drug eluting members 74 extending along a length of the elongate expandable body 72. Each of the drug eluting members 74 may be considered as being examples of the drug eluting member 28, for example. While a total of three drug eluting members 74 are shown, it will be appreciated that this is merely illustrative.

The implantable medical device 70 may include any number of drug eluting members 74, for example. Each of the drug eluting members 74 may include the same one or more therapeutic drugs. In some cases, one of the drug eluting members 74 may include a first therapeutic drug and another of the drug eluting members 74 may include a second therapeutic drug.

In some cases, it is contemplated that the drug eluting members 74 may be provided with the therapeutic drugs already provided in or on each of the drug eluting members 74. Depending on what the implantable medical device 70 is, and perhaps where it is being implanted, the physician or other professional implanting the implantable medical device 70 may select particular drug eluting members 74, each loaded with one or more particular therapeutic drugs, for implantation along with the elongate expandable body 72.

In some cases, the implantable medical device 70, including both the elongate expandable body 72 and the drug eluting members 74, may be implanted using the delivery device 10 (FIG. 1), with the elongate expandable body 72 and the drug eluting members 74 kept separate until deployment. In some cases, the implantable medical device 70, including both the elongate expandable body 72 and the drug eluting members 74, may be delivered by loading together the elongate expandable body 72 and the drug eluting members 74 into a delivery device that is adapted to deliver both the elongate expandable body 72 and the drug eluting members 74 at the same time.

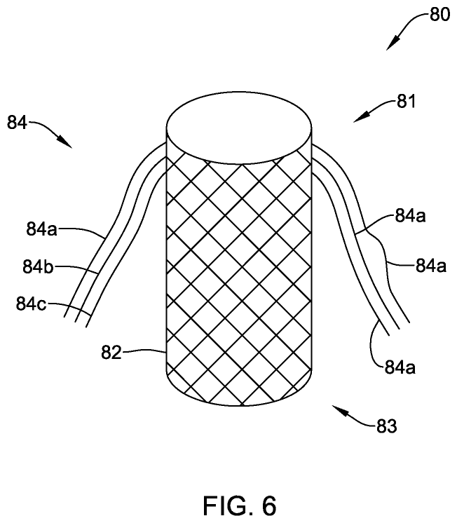

In some cases, the drug eluting members 74 may be secured in place relative to the elongate expandable body 72 as a result of the drug eluting members 74 being pinned between the elongate expandable body 72 and the side wall of the lumen in which the implantable medical device 70 is being deployed. As the elongate expandable body 72 expands from a collapsed or compressed configuration for delivery into an expanded configuration for deployment, the elongate expandable body 72 expands such that the elongate expandable body 72 contacts the side wall of the lumen in which the implantable medical device 70 is being deployed. In some cases, the drug eluting members may instead be secured to the elongate expandable body, as shown for example in FIG. 6.

FIG. 6 is a side view of an illustrative implantable medical device 80. The implantable medical device 80 includes an elongate expandable body 82 that is moveable between a compressed configuration for delivery (not shown) and an expanded configuration, as illustrated. The elongate expandable body 82 extends from a distal region 81 to a proximal region 83. In some cases, a number of drug eluting members 84 may be secured relative to the distal region 81 of the elongate expandable body 82. The drug eluting members 84 may be adhesively secured relative to the elongate expandable body 82, for example, or may be welded or soldered in place.

As shown, several drug eluting members 84a are secured along a first side of the distal region 81 of the elongate expandable body 82 and several drug eluting members 84b are secured along a second side of the distal region 81 of the elongate expandable body 82. The drug eluting members 84a and 84b extend proximally from where they are attached. While shown spaced apart from the elongate expandable body 82, this is merely for clarity. In use, particularly once the elongate expandable body 82 has expanded into its expanded configuration, the drug eluting members 84a and 84b will be pressed between the elongate expandable body 82 and a side wall of the lumen in which the implantable medical device 80 is deployed.

Each of the drug eluting members 84 may be considered as being examples of the drug eluting member 28, for example. While a total of three drug eluting members 84 are shown, it will be appreciated that this is merely illustrative. The implantable medical device 80 may include any number of drug eluting members 84, for example. Each of the drug eluting members 84 may include the same one or more therapeutic drugs. In some cases, one of the drug eluting members 84 may include a first therapeutic drug and another of the drug eluting members 84 may include a second therapeutic drug. In some cases, it is contemplated that the drug eluting members 84 may be provided with the therapeutic drugs already provided in or on each of the drug eluting members 84.

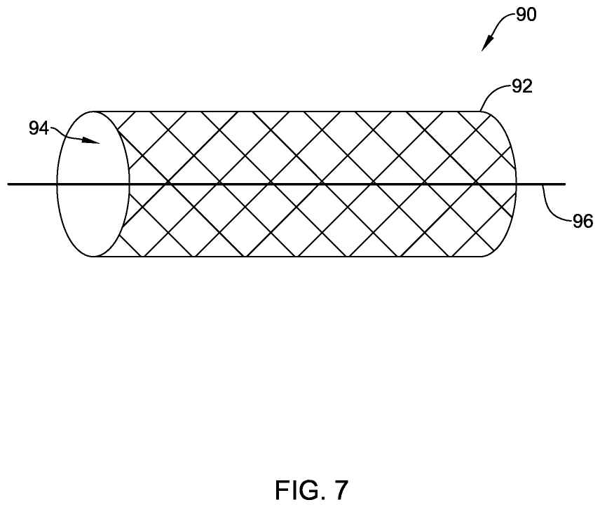

FIG. 7 is a side view of an illustrative implantable medical device 90. The implantable medical device 90 includes an elongate expandable body 92 that is moveable between a compressed configuration for delivery (not shown) and an expanded configuration, as illustrated. The elongate expandable body 92 defines an interior 94 extending through the elongate expandable body 92. A drug eluting member 96 extends through the interior 94 of the elongate expandable body 92.

The drug eluting member 96 may be considered as being example of the drug eluting member 28, for example. While only one drug eluting member 96 is shown, it will be appreciated that this is merely illustrative. The implantable medical device 90 may include any number of drug eluting members 96.

In some cases, the implantable medical device 90, including both the elongate expandable body 92 and the drug eluting member 96 may be delivered by loading together the elongate expandable body 92 and the drug eluting member 96 into a delivery device that is adapted to deliver both the elongate expandable body 92 and the drug eluting member 96 at the same time.

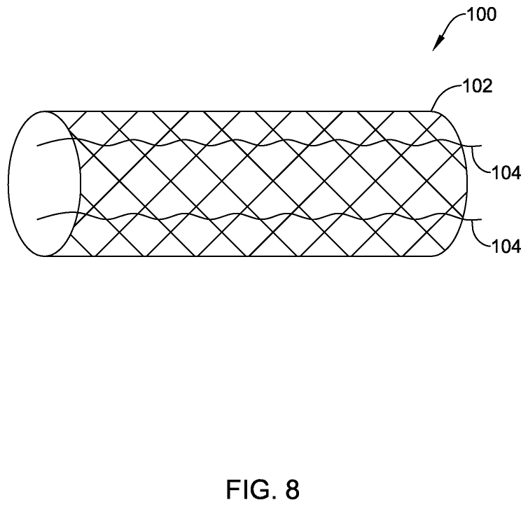

FIG. 8 is a side view of an illustrative implantable medical device 100. The implantable medical device 100 includes an elongate expandable body 102 that is moveable between a compressed configuration for delivery (not shown) and an expanded configuration, as illustrated. A pair of drug eluting members 84 are each woven into the elongate expandable body 102.

Each of the drug eluting members 104 may be considered as being example of the drug eluting member 28, for example. While two drug eluting members 104 are shown, it will be appreciated that this is merely illustrative. The implantable medical device 100 may include any number of drug eluting members 104.

In some cases, the implantable medical device 100, including both the elongate expandable body 102 and the drug eluting members 104 may be delivered by loading together the elongate expandable body 102 and the drug eluting members 104 into a delivery device that is adapted to deliver both the elongate expandable body 102 and the drug eluting members 104 at the same time.

Each of the drug eluting members 104 may include the same one or more therapeutic drugs. In some cases, one of the drug eluting members 104 may include a first therapeutic drug and another of the drug eluting members 104 may include a second therapeutic drug.

In some cases, it is contemplated that the drug eluting members 104 may be provided with the therapeutic drugs already provided in or on each of the drug eluting members 104. Depending on what the implantable medical device 100 is, and perhaps where it is being implanted, the physician or other professional implanting the implantable medical device 100 may select particular drug eluting members 104, each loaded with one or more particular therapeutic drugs, for implantation along with the elongate expandable body 102.

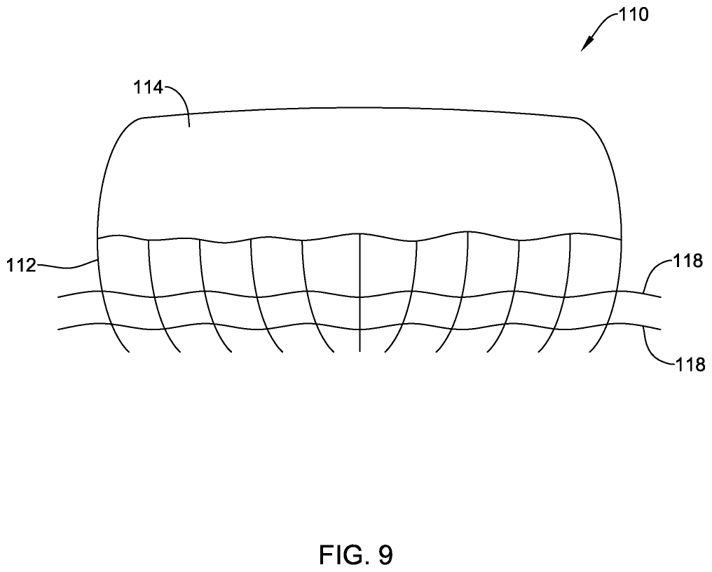

FIG. 9 is a side view of an illustrative implantable medical device 110. While some of the implantable medical devices shown in the previous drawings are stents, it will be appreciated that the implantable medical device 110 is a cardiac implant. In particular, the implantable medical device 110 may be considered as being a Left Atrial Appendage (LAA) closure device. The implantable medical device 110 includes an expandable framework 112 and a fabric covering 114 that covers a portion of the expandable framework 112.

In some cases, one or more drug eluting members 118 may be woven into the expandable framework 112. While two drug eluting members 118 are shown as being woven into the expandable framework 112, it will be appreciated that in some cases there be three or more drug eluting members 118 woven into the expandable framework 112. Each of the drug eluting members 118 may include the same one or more therapeutic drugs. In some cases, one of the drug eluting members 116 may include a first therapeutic drug and another of the drug eluting members 118 may include a second therapeutic drug.

In some cases, it is contemplated that the drug eluting members 118 may be provided with the therapeutic drugs already provided in or on each of the drug eluting members 118. Depending on particular needs of the patient, the physician or other professional implanting the implantable medical device 110 may select particular drug eluting members 118, each loaded with one or more particular therapeutic drugs, for implantation. It will be appreciated that once the implantable medical device 110 has been implanted, the drug eluting members 118 may be captured between the expandable framework 112 and the tissue surrounding the expandable framework 112. In some cases, at least some of the drug eluting members 118 may be secured to the expandable framework 112 via any appropriate technique including adhesives, soldering and welding.

The therapeutic agents used in forming the drug eluting members 28, 74, 84, 96, 104, 116 and 118 may include one or more of anti-thrombogenic agents and/or anticoagulants such as heparin, coumadin, heparin derivatives, urokinase, and PPack (dextrophenylalanine proline arginine chloromethylketone) D-Phe-Pro-Arg chloromethyl keton, an RGD peptide-containing compound, antithrombin compounds, platelet receptor antagonists, anti-thrombin antibodies, antiplatelet receptor antibodies, aspirin, prostaglandin inhibitors, platelet inhibitors, and tick antiplatelet peptides; anti-proliferative agents such as enoxaprin, angiopeptin, or monoclonal antibodies capable of blocking smooth muscle cell proliferation, hirudin, and acetylsalicylic acid; anti-inflammatory agents such as dexamethasone, prednisolone, corticosterone, budesonide, estrogen, sulfasalazine, and mesalamine; antineoplastic/antiproliferative/anti-miotic agents such as paclitaxel, 5-fluorouracil, cisplatin, vinblastine, vincristine, epothilones, endostatin, angiostatin and thymidine kinase inhibitors; anesthetic agents such as lidocaine, bupivacaine, and ropivacaine; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; anti-sense DNA and RNA; and DNA coding for (and the corresponding proteins) anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin like growth factor, cell cycle inhibitors including CD inhibitors, thymidine kinase (“TK”) and other agents useful for interfering with cell proliferation, and the family of bone morphogenic proteins (“BMP's”) including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, BMP-16, “hedgehog” proteins.

The devices discussed herein, and various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A delivery device adapted to deliver an implantable medical device to a deployment site, the delivery device comprising:
an elongate shaft extending from a proximal region to a distal region, the elongate shaft defining:
a first portion adapted to accommodate the implantable medical device; and
a second portion adapted to accommodate a drug eluting component to be delivered along with the implantable medical device;
wherein the delivery device is adapted to release the implantable medical device at the deployment site.

2. The delivery device of claim 1, wherein the second portion comprises a passageway in fluid communication with a source of an elutable drug.

3. The delivery device of claim 2, wherein the delivery device is adapted to bathe the implantable medical device with the elutable drug prior to releasing the implantable medical device.

4. The delivery device of claim 2, wherein the delivery device is adapted to bathe the deployment site with the elutable drug either before, during or after releasing the implantable medical device.

5. The delivery device of claim 1, wherein the second portion keeps the drug eluting component away from the implantable medical device until deployment.

6. The delivery device of claim 5, wherein a sheath separates the first portion from the second portion until the sheath is retracted.

7. The delivery device of claim 1, wherein the drug eluting component comprises one or more elongate members carrying an elutable drug.

8. The delivery device of claim 7, wherein the one or more elongate members comprise threads or wires that are impregnated or coated with the elutable drug.

9. An implantable medical device, comprising:
a body expandable from a compressed delivery configuration to an expanded deployment configuration; and
one or more drug eluting components that are formed separately from the body and disposed proximate the body upon deployment of the implantable medical device.

10. The implantable medical device of claim 9, wherein the one or more drug eluting components are woven into the body.

11. The implantable medical device of claim 9, wherein the one or more drug eluting components are dispersed over an exterior of the body.

12. The implantable medical device of claim 9, wherein the one or more drug eluting components are disposed proximate the body after the body has been compressed into its compressed delivery configuration.

13. The implantable medical device of claim 9, wherein the one or more drug eluting components are delivered proximate the body after the body has been delivered and before the body has expanded into the expanded deployment configuration.

14. The implantable medical device of claim 9, comprising a stent.

15. The implantable medical device of claim 9, comprising a cardiac implant.

16. A bare metal stent, comprising:
an expandable elongate body expandable from a compressed delivery configuration to an expanded deployment configuration; and
one or more drug eluting components that are formed separately from the expandable elongate body and disposed proximate the expandable elongate body upon deployment of the bare metal stent.

17. The bare metal stent of claim 16, wherein the one or more drug eluting components are woven into the expandable elongate body.

18. The bare metal stent of claim 16, wherein the one or more drug eluting components are dispersed over an exterior of the expandable elongate body.

19. The bare metal stent of claim 16, wherein the one or more drug eluting components are disposed proximate the expandable elongate body after the expandable elongate body has been compressed into its compressed delivery configuration.

20. The bare metal stent of claim 16, wherein the one or more drug eluting components are delivered proximate the expandable elongate body after the expandable elongate body has been delivered and before the expandable elongate body has expanded into the expanded deployment configuration.
